(19) United States
(12) Patent Application Publication
(10) Pub. No.: US 2023/0282332 A1
(43) Pub. Date: Sep. 7, 2023

(54) SYSTEM AND METHOD FOR REAL-TIME PROVIDING OF PRACTICE RECOMMENDATIONS BASED ON BARRIERS TO CLIENT ENGAGEMENT

(71) Applicant: RAMBAM MED-TECH LTD., Haifa (IL)

(72) Inventor: Goded SHAHAF, Haifa (IL)

(21) Appl. No.: 18/008,169

(22) PCT Filed: Jun. 6, 2021
(86) PCT No.: PCT/IL2021/050676

§ 371 Date: Dec. 4, 2022

Related U.S. Application Data
(60) Provisional application No. 63/191,340, filed on May 21, 2021.
(60) Provisional application No. 63/034,990, filed on Jun. 5, 2020.

(51) Int. Cl. G16H 20/70; A61B 5/369; A61B 5/11; G16H 50/30

(52) U.S. Cl. CPC G16H 20/70; A61B 5/369; A61B 5/1103; G16H 50/30

(57) ABSTRACT

System and methods are adapted to extract client attention/engagement/effort barrier electrophysiological signal during practice, to combine the attention/engagement/effort barrier with level of success of the client in performing practice task, in order to provide practice recommendation during the current session or following it.

### CROSS-REFERENCE TO RELATED APPLICATIONS

This application claims the benefit of priority to U.S. Provisional Patent Application Nos. 63/034,990, filed Jun. 5, 2020, and 63/191,340 filed May 21, 2021, both entitled “Finding affective+cognitive barriers to performance by combining effort dynamics”, the contents of which are incorporated herein by reference in their entirety.

### BACKGROUND OF THE INVENTION

Rehabilitation treatment for a patient suffering from, for example, brain or peripheral injury, or learning for a student with learning disabilities may achieve reduced effect due to limited participation of the client in the process. There is ample evidence that an active participation of a client, by means of exerting deliberate effort to work towards achieving the rehabilitation goal (or, in short, the client engagement), is of major clinical importance. Better outcome with effective engagement was consistently reported in rehabilitation and education.

It has been suggested that client engagement may lead to greater brain (or neural) activity in the relevant brain regions, and thereby to greater plasticity, both are highly required processes. Nevertheless, it seems that at certain times in the rehabilitation and learning processes reduced, yet focused, activity would be more effective in obtaining better outcome. Thus, it is not always the case that global increase in brain activity would be contributive, but rather focused activation of certain brain pathways and representations. In this case, greater engagement might lead to better selective and/or sustained attention, rather than to overall greater neural recruitment. Furthermore, in other conditions, such as rehabilitation for chronic pain or psychiatric rehabilitation e.g. for anxiety disorders, it actually seems that reduction in global activity, with focused activation of certain pathways and representations, would be preferable. Yet, the importance of client engagement was also demonstrated in these conditions. Thus, it is not necessarily greater overall neural activation and greater overall plasticity, which are induced by effective client engagement, but, possibly, rather better selective and sustained attention focused upon the rehabilitation and learning goals and exercises.

The literature describes two types of constitutional conditions which hinder the client's ability to obtain effective engagement: (1) dysfunctional affective coping and (2) limited cognitive recruitment and specifically attention deficit. Both the affective and cognitive problems could be premorbid or newly acquired due to neurological injury, which necessitated rehabilitation in the first place. Notably, both cognitive/attentional and affective disorders hinder attention, which accords with the identification of attention processes as the neuropsychological mechanism, which underlies client engagement.

A clearly pathological constitutional condition (either cognitive or affective and either premorbid or newly acquired) of the client could certainly lead to ineffective engagement. However, often it might be that the client's cognitive and affective status is not necessarily globally dysfunctional, but rather that certain exercises, during the rehabilitation session, might be too demanding cognitively, or too threatening. For example, a post-stroke patient may feel insecure with regard to standing or walking again after the injury, even if their basic ability to do this is relatively preserved. For another example, it might be that for a post-stroke patient the ability to focus sufficiently on the task of recalling words might be too demanding, even if their basic ability is relatively preserved. Generally, cognitive and affective barriers evoked by specific exercises during the rehabilitation or learning session may be very prevalent, even when the basic cognitive and affective status of the client is relatively preserved.

Thus, there are two layers of engagement evaluation—the basic layer, which is more constitutional and relates to the general cognitive and affective status of the client, and the situational layer, which relates to the impact of exercise selection and implementation upon the transient client engagement. Therefore, beyond acquaintance with the client's basic engagement level, it seems important for the therapist/teacher/trainer to monitor for cognitive and affective barriers which may hinder client engagement during specific exercises and to intervene accordingly during the session.

Multiple easy-to-use electrophysiological markers for attention, engagement or effort have been suggested over the recent years for multiple indications, as an assistive tool to improve analysis of the client's performance on the basis of her overt behavior. However, no systematic way was offered to combine the electrophysiological markers with the behavioral performance, in the various fields of implementation—such as during a client's training sessions, during a client's evaluation sessions, during the evaluation of media exposure, etc. The current application describes a system, which interprets automatically the interaction between the behavioral performance and the dynamics of the electrophysiological markers for attention in each one of these session types.

There is a need for a system, which analyzes the interaction of electrophysiological markers for attention, engagement or effort (out of multiple candidate attention markers), on the one hand, and evaluation of behavioral performance, on the other hand. The dynamics of the attention marker over time (at-least a few tens of seconds) needs to be analyzed to identify cognitive or affective barriers in the exercise performed, in a manner, which is described in detail below.

### SUMMARY OF THE INVENTION

A method for providing practice (training, evaluation or media exposure) recommendations during or following a practice session is disclosed comprising receiving at least one electrophysiological signal of a client from an EEG system or an eye tracking system during the practice session, receiving indication of the success of the client in performing a task during the practice session, extracting electrophysiological markers for attention/engagement/effort of the client during the performance of the task, extracting client engagement barrier types from the electrophysiological markers, classifying client engagement barrier types to one of: affective barrier, cognitive barrier and no barrier, classifying the success level of the client in performing the task to one of a plurality of discrete success levels and providing practice recommendation for the current or for future practice based on the specific success level and on the identified attention barrier.

In some embodiments the plurality of discrete success levels comprise: low performance, moderate performance and high performance.

In some embodiments the extracting of client engagement barriers from the electrophysiological markers comprises extraction of an attention/engagement/effort index.

In some embodiments the extraction of an attention/engagement/effort index comprises dividing the electrophysiological signal into a plurality of segments and dividing each of the segments into a plurality of epocs.

In some embodiments the duration of each of the plurality of the segments is in the range of seconds to tens of seconds and the duration of each of the epocs is in the range of hundreds of milliseconds to seconds. In some embodiments the duration of each of the plurality of the segments is 10 seconds and the duration of each of the epocs is 500 milliseconds.

In some embodiments the method further comprising excluding epocs in which the signal deviation is above a predefined level, to remove noisy epocs.

In some embodiments the method further comprising assigning power index to each of the remaining epocs according to the average absolute amplitude of the signal in each epoc of the remaining epocs and normalizing the power index to a normalized range.

In some embodiments the method further comprising identifying attention barrier type associated with the received signal based on normalized power index dynamics and the relation between the normalized power indices to a lower threshold and to a higher range in the normalized range.

A system for providing practice recommendations during or following a practice session, the system comprising a computing unit adapted to receive at least one electrophysiological signal of a client from an EEG system or an eye tracking system during the practice session and indication of the success of the client in performing a task during the practice session, the computing unit comprising a central processing unit (CPU), a memory unit, a non-transitory storage unit and an input/output unit, wherein the CPU is adapted to perform executable code loadable from the memory unit and/or the storage unit, wherein the input unit is adapted to receive the at least one electrophysiological signal of a client from an EEG system during the practice session and the indication of the success of the client in performing the task during the practice session and the output unit is adapted to provide practice recommendations based on the received one electrophysiological signal of a client from an EEG system during the practice session and received indication of the success of the client.

In some embodiments the system is further adapted to extract electrophysiological markers for attention of the client during the performance of the task, to extract client engagement barrier types from the electrophysiological markers, to classify client engagement barrier types to one of: affective barrier, cognitive barrier and no barrier, to classify the success level of the client in performing the task to one of a plurality of discrete success levels and to provide practice recommendations for a future practice based on the specific success level and on the identified attention barrier.

In some embodiments the plurality of discrete success levels comprise: low performance, moderate performance and high performance.

In some embodiments the extracting of client engagement barriers from the electrophysiological markers comprises extraction of an attention/engagement/effort index.

In some embodiments the extraction of an attention/engagement/effort index comprises dividing the electrophysiological signal into a plurality of segments and dividing each of the segments into a plurality of epocs.

In some embodiments the duration of each of the plurality of the segments is in the range of seconds to tens of seconds and the duration of each of the epocs is in the range of hundreds of milliseconds to seconds. In some embodiments the duration of each of the plurality of the segments is 10 seconds and the duration of each of the epocs is 500 milliseconds.

In some embodiments the system further comprising excluding epocs in which the signal deviation is above a predefined level, to remove noisy epocs.

In some embodiments the system further comprising assigning power index to each of the remaining epocs according to the average absolute amplitude of the signal in each epoc of the remaining epocs and normalizing the power index to a normalized range.

In some embodiments the system further comprising identifying attention barrier type associated with the received signal based on normalized power index dynamics and the relation between the normalized power indices to a lower threshold and to a higher range in the normalized range.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 demonstrates extraction of CEI index from an EEG signal, according to embodiments of the present invention;

FIGS. 2A-2D demonstrate the patterns of dynamics in an index of attention over time, according to embodiments of the present invention;

FIG. 2E illustrates how high variability among the 500 milliseconds epocs of the 10-second segments yields a higher CEI value in comparison with a lower variability segment, according to embodiments of the present invention;

FIG. 3 presents a screenshot of a practice recommendation for moderate end-of-session performance and an affective barrier, according to embodiments of the present invention;

FIG. 4A presents the attention index dynamics during a session. According to embodiments of the present invention;

FIG. 4B presents the attention index dynamics during the client's session, according to embodiments of the present invention;

FIG. 5A presents the attention index dynamics of the session, according to embodiments of the present invention;

FIG. 5B presents the attention index dynamics of the session, according to embodiments of the present invention;

FIG. 5C presents the attention index dynamics during the session, according to embodiments of the present invention;

FIG. 6A presents the attention index dynamics during the session, according to embodiments of the present invention;

FIG. 6B presents the attention index dynamics during the session, according to embodiments of the present invention;

FIG. 6C presents the attention index dynamics of the session according to embodiments of the present invention;

FIGS. 7A and 7B present schematic block diagram of a system for real-time monitoring of barriers to client engagement and of a computing unit adapted to compute the barriers and to provide practice recommendation, respectively, according to embodiments of the present invention; and

FIG. 8 is a schematic flow diagram depicting a method for real-time monitoring barriers to client's engagement and for providing practice recommendations, according to embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

As mentioned, client engagement is based upon the neuropsychological process of sustained attention. Multiple electrophysiological markers have been suggested for attention and there is ample literature considering electrophysiological markers for attention and their change with affective dysfunction and with cognitive dysfunction. The electrophysiological markers for attention are reduced with cognitive impairment and in avoidance/self-inhibition—for example in depressive states. On the other hand, these markers might be enhanced in various anxious states. However, the attention markers discussed in the literature are generally not simple to extract and are, often, not extractable at all in real-time at the sub-minute timescale.

Embodiments of the present invention enable to extract such electrophysiological markers for attention from very simple and easy to use EEG systems (such as the NeuroSky MindWave) or EMG system or eye tracking system. These extracted markers tend to be low with cognitive and attention deficits, tend to be low with depression and tend to be high in anxious and stressful conditions. Furthermore, these markers have been shown to tend toward the middle range with effective client engagement. Vast practical experience was obtained with the use of these markers, including an advanced CEI (Cognitive Effort Index) marker, for monitoring client engagement in rehabilitation and learning with variable clinical and non-clinical populations, including also patients undergoing physical rehabilitation, patients undergoing cognitive and language rehabilitation, patients with severe pain syndromes, patients with severe disorders of consciousness and patients undergoing psychiatric rehabilitation. The description below refers, in general, to the computation of an attention index. In some of the described examples the general attention index is exemplified by a cognitive effort index (CEI), yet it would be apparent to those skilled in the art that the examples using the CEI may be utilized for computing other attention indices. Further, the term ‘practice recommendation’ and the term ‘treatment recommendation’ may be used interchangeably in the description of embodiments of the invention herein below.

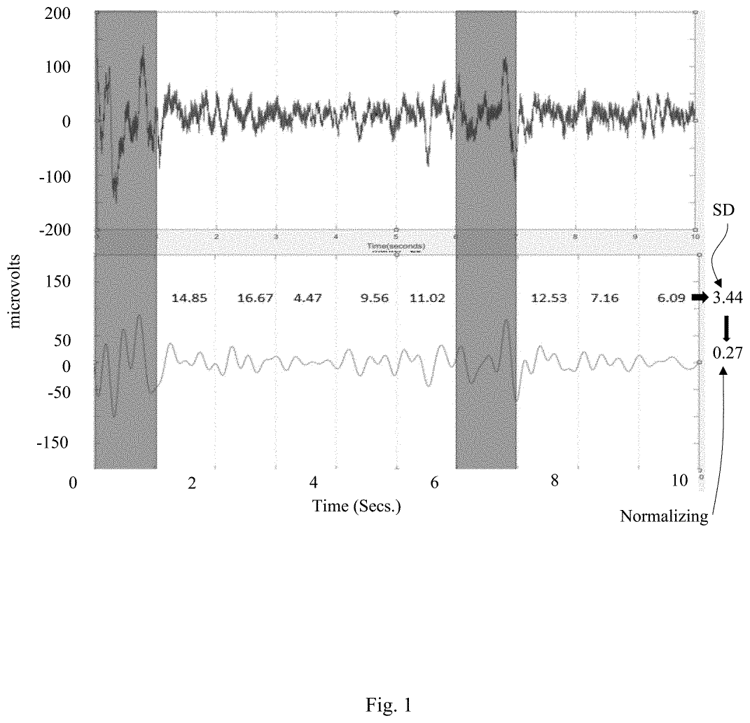

Reference is made now to FIG. 1, which demonstrates extraction of CEI index from an EEG signal, according to embodiments of the present invention. The data may be analyzed, online or offline, in segments of a predefined duration—e.g. 10 seconds, as presented in the top graph. Each segment may be divided to epocs of a predefined duration—e.g. 1 second. Noisy epocs are excluded—e.g. due to deviant amplitude as marked in the grey-shaded bands. Data may be filtered for a given band—e.g. Delta—1-4 Hz, as presented in the bottom graph. For the remaining valid epocs, a power index may be computed, or in this specific case—average absolute amplitude in the delta activity band. The resulting values are presented numerically for each epoc in the bottom graph (e.g. 14.85 for the second epoc, 16.67 for the third epoc, etc.). The standard deviation of these values may be computed as pointed at by the horizontal arrow. This value then may be normalized based on previous samples, as presented by the vertical arrow (division by 12.5 in this example) to generate the attention index.

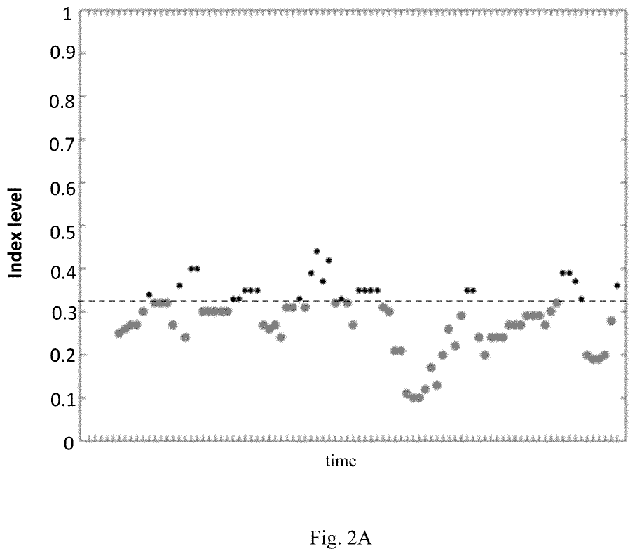

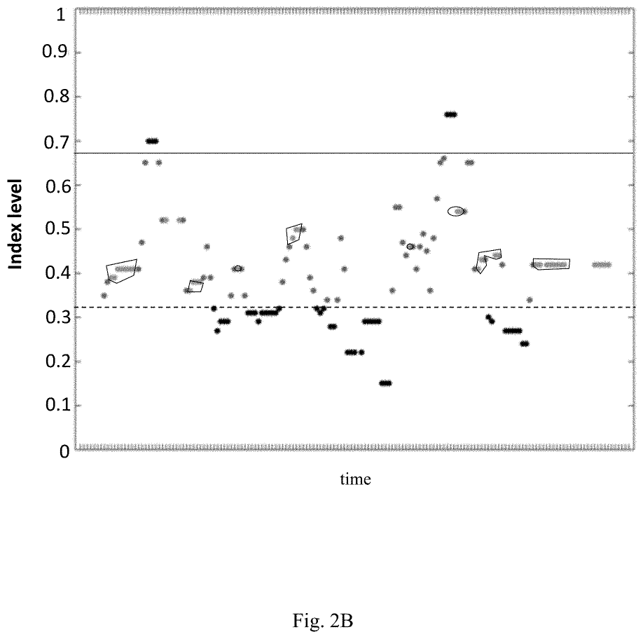

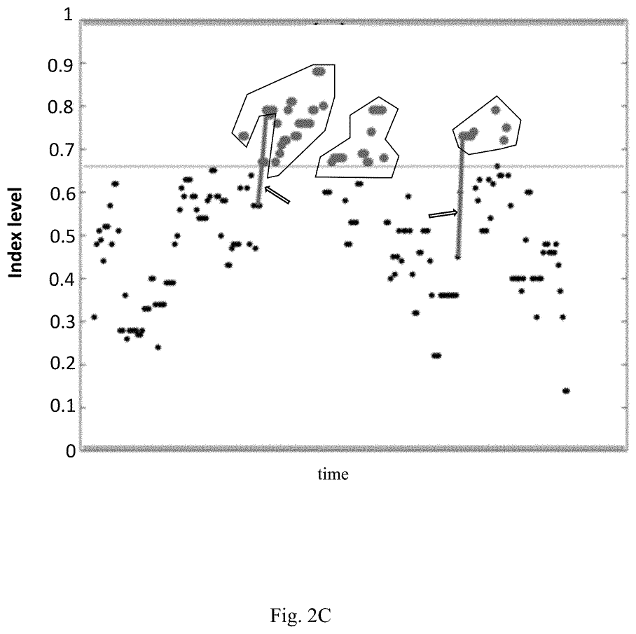

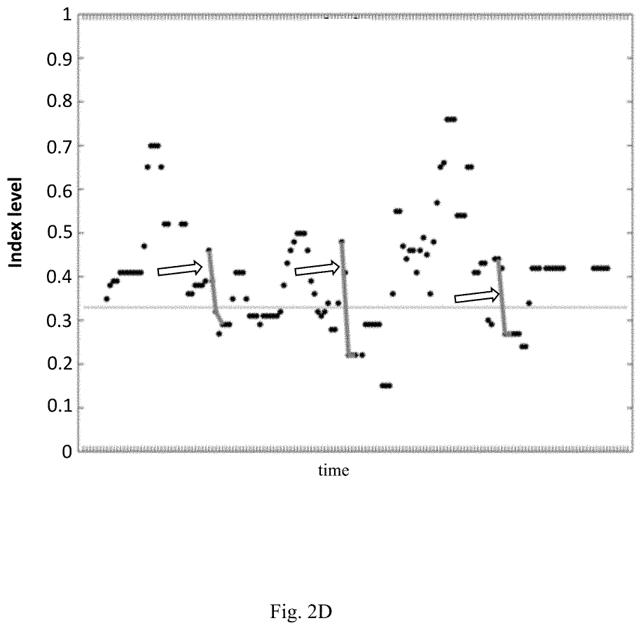

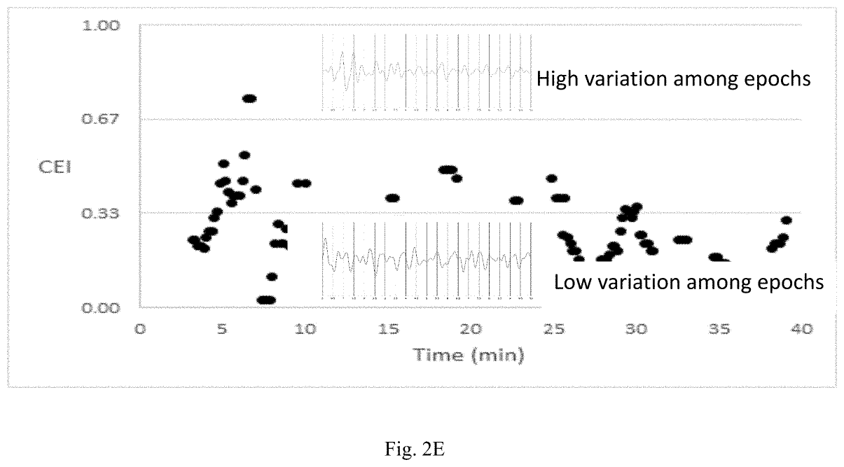

Reference is made now to FIGS. 2A-2D, which demonstrate the patterns of dynamics in an index of attention over time, according to embodiments of the present invention. Each point in FIGS. 2A-2D summarizes the attention marker over 10 seconds. The marker is shown at a normalized [0,1] range, where higher index indicates greater attention. Any possible attention marker (some examples follow) is valid for the analysis of the pattern of dynamics over time. FIG. 2A includes grey points, which are below a lower (e.g. ⅓) threshold and black points, which are above this threshold. It is possible to note that the majority of points in the sample are below ⅓ (grey) and therefore the pattern is identified as pattern A described below (low attention).

FIG. 2B includes “red” points (points above the line that are not encircled) and “green” points above the line, encircled), which are above a ⅓ threshold and below a ⅔ threshold and black points, which are outside this range. The difference between the “red” and “green” points is that the green point values are similar to the values of their preceding and succeeding points (i.e. low variability between successive points) and thus denote a stable attention level. Altogether, it is possible to see that the “red” and “green” points form the majority of points in the sample and therefore the pattern is identified as pattern B described below (intermediate attention).

FIG. 2C shows “red” points (points encircled by black line), which are above a ⅔ threshold and black points, which are below ⅔. It also shows the ⅔ threshold with an horizontal line and marks by black arrows two times where there is a sharp upslope, which crosses this threshold—with an upslope of at-least 0.1 within 20-30 seconds. Such findings are associated with patterns C and D described below (high attention and sharp increases respectively).

FIG. 2D shows the ⅓ threshold with an horizontal line and marks by black arrows three times where there is a sharp downslope, which crosses this threshold—with a downslope of at-least 0.1 within 20-30 seconds. Such findings are associated with pattern E below (sharp decreases).

Interpretation of interactions of barriers and behavioral performance is presented in Table 1, below. The barrier may be identified from the pattern of dynamics in the electrophysiological marker of attention (as is presented below).

In the discussion below attention markers are widely discussed and described, yet it would be apparent that engagement markers and/or effort markers may also be used for embodiments of the current invention. Some markers for attention are extractable from one electrophysiological channel or more (e.g. power and power ratio based markers, template-matching based markers, variability patterns and blink patterns), while for others at-least two electrodes might be required (e.g. synchronization based markers). The electrophysiological channels are comprised of a target electrode and of a reference electrode (or a combination of electrodes)—The reference electrodes may be shared by multiple target electrodes, as is known in the art. The electrodes for the various markers may be placed all over the head. Preferably, the electrodes may be placed below the hairline, directly on the skin, so as to receive a good signal with greater ease. The electrodes may also be placed in the ears, or in the ears vicinity. Multiple studies have shown the ability to extract an effective attention related marker from below the hairline and mainly from the forehead.

Various methods are employed in the literature to identify in real-time, at a temporal resolution of a few seconds, a marker for attention and any of these (and additional) methods may be used for the purpose of the current system. Some examples for this ability are described herein below.

A. Power and Power Ratio Based Markers: It was found that greater attention often involves greater activity power in higher EEG frequency bands (e.g. beta—˜13 Hz-˜30 Hz) and reduced attention often involves greater activity power in lower frequency bands (e.g. theta—˜4 Hz-˜7 Hz). Activity power could be computed in multiple ways—e.g. Fourier-based power analysis, integral of absolute values, etc. Furthermore, it is customary to compute the ratio between a lower frequency (e.g. theta) and the higher frequency (e.g. beta) as an indication of lower attention (and vice versa).

B. Synchronization Based Markers: It was found that greater attention often involves synchronization between distant electrodes for some EEG frequency bands (e.g. theta—˜4 Hz-˜7 Hz, beta—˜13 Hz-˜30 Hz and gamma—>˜30 Hz) and desynchronization for other frequency bands (e.g. alpha—˜8 Hz-˜12 Hz). The synchronization/desynchronization could be computed between any two electrophysiological channels on the head—whether ipsilateral (on the same side) or contralateral.

Synchronization can be computed in various methods. It may be based on any correlation index between the simultaneous sampling points of the contralateral channels—for example Pearson correlation or coherence analysis. Furthermore, it is possible to transform the raw signals before the synchrony analysis—for example to look at synchronization in specific frequency bands—e.g. delta, theta, alpha, beta or gamma—or their combination. It is also possible to analyze synchronization between transformations of the raw signals by template matching, wavelet analysis and components analysis. Synchronization can also be computed on the basis of the ratio between the level of activity on both sides, or their power in any frequency band, or combination of frequency bands.

It is also possible to transform the signal into discrete values and to evaluate correlation between these discrete values. For one example, it is possible to transform the signal to positive and negative deflections from the average during the selected timeframe and compute the hamming distance as an index for correlation. But it is also possible to divide the signal into more discrete values.

C. Template-Matching Based Markers: Multiple pattern templates were identified as related to attention. These templates could involve any combination of frequency bands and wave patterns. They could be derived from data mining methods, which identify them as related with an experimental condition, which is expected to involve attention. See for example: Monitoring attention in ADHD with an easy-to-use electrophysiological index. At times, they are derived from association to previously identified markers in EEG or in other modalities.

These markers are then sought in the sampled electrophysiological activity. Their matching with the sampled activity might be based on various methods of signal correlation—such as Fourier-based correlation and computation of average distance between the template and a matched-size moving window in the sampled data. The matching could be done after amplitude normalization of both the template and moving window to an equivalent range. The distance evaluation could be done by computation of the average distance over sampling points, or by any other accepted method of distance evaluation between two sampled signals.

D. Variability Based Markers: It has been shown that rapid signal variability between consecutive sample sequences, which last between tens of milliseconds to few seconds, associate with level of attention. For some sequence patterns it seems that reduced inter-sequence variability is associated with a higher level of attention, while for other sequence patterns it seems that increased inter-sequence variability is associated with higher level of attention. This duality stems from a set of factors, such as the frequency band of the activity, which corresponds well with the fact that the activity of some frequency bands increases with attention, while the activity of others decreases. Other factors may include spatial location of the electrophysiological channel etc.

For the analysis of variability, a quantification of each of the consecutive segments could be generated, such as segment's average power, segment's standard deviation, segment's max or min amplitude, segment's power analysis in various frequency band, degree of occurrence of a certain template in the segment, etc. Then variance between the set of segments could be computed—e.g. by variance analysis, standard deviation analysis, inter-percentile distance, etc. Alternatively, it is possible to evaluate the distance of each segment from a template and to compute the average distance among these evaluations, or some other group summary.

Thus, it is possible to compute variability based on a quantification of each segment or based on a distance from a given predetermined value. Either way, the quantifying value of each segment could be normalized to a pre-determined range (e.g. [0,1]) before computing the overall variability.

E. Blink Based Markers: It has been shown that blinking is associated with attention. Generally, slower blinking is associated with increased attention. Blinks are recordable from the electrophysiological channels as well as from eye movement sensors. The blinks are identifiable by analysis of large slow frequency (e.g. in the delta range) deflections of more than few tens of microvolts (e.g. more than 30 microvolts) with gaps of few hundred milliseconds (e.g. at-least 500 milliseconds from a previous blink identification). The frequency of the blinks is then calculated as an index for attention. For example, increased attention might be related to a blinking frequency of ˜¼ Hz and decreased attention might be related to a blinking frequency of ˜½ Hz. Specifically, for blinking, it is also possible to extract them from eye tracker blink detectors.

For all the above types of markers as well as for other electrophysiological markers for attention, prior to the computation of the marker, it is possible to remove from the signal epocs of definite size (e.g. from parts of seconds to a few seconds), which are identified as noisy—e.g. due to large-amplitude waves, or by any other means, or to filter out such epocs—e.g. ECG and only thereafter to compute the index. Then, it is possible to generate a marker only for samples with enough epocs (e.g. more than 50% valid epocs). It is also possible to include in the analysis only specific types of filtered activity—e.g. specific frequency bands, specific wavelets, specific principal or independent components, etc. Finally, it is possible to normalize whichever selected marker to a set range—e.g. [0,1]. FIG. 1 presents one example of an attention marker computation. In this example the computation is variability based, but, as stated above, the index can be computed using multiple methods, as detailed in the section above and in the literature.

For any selected period of time, from a few tens of seconds and above, and for any selected attention marker from the list above, it is possible to identify several dominant patterns, which may include:

A. Low level of the attention marker over the majority of the sampled period: when the majority of attention marker values during the sampled period are below the lower threshold (e.g. ˜⅓ in the normalized [0,1] scale). See for example FIG. 2A. B. Intermediate level of the attention marker over the majority of the sampled period: when the majority of attention marker values during the sampled period are between the lower threshold (e.g. ˜⅓ in the normalized [0,1] scale) and the higher threshold (e.g. ˜⅔ in the normalized [0,1] scale). See for example FIG. 2B. C. High level of the attention marker over the majority of the sampled period: when the majority of attention marker values is above the higher threshold (e.g. ˜⅔ in the normalized [0,1] scale). See for example FIG. 2C—“red” dots. D. Sharp increases of the attention marker over few tens of seconds: when there are increases from below to above the higher threshold during the sampled period and the majority of these increases have an upslope of more than ˜0.1 in 10-30 seconds. See for example FIG. 2C—“red” upslope; and E. Sharp decreases of the attention marker over few tens of seconds: when there are decreases from above to below the lower threshold during the sampled period and the majority of these decreases have a downslope of more than ˜0.1 in 10-30 seconds. See for example FIG. 2D.

Patterns A, B and C, on the one hand, and patterns D and E, on the other hand, are not mutually exclusive. Therefore, it is possible to define a preference between them—for example that if patterns D or E occur they are identified and only if they do not occur, patterns A, B or C are identifiable (or vice-versa).

The interpretation of the electrophysiological markers with regard to the client's engagement depends upon client performance. When the client performance is not improving over the session, it might be due to barriers in engagement, which are expected to manifest in the marker. However, when the client reaches major improvement during the session and this is done with ease and low allocation of attention, the marker is also expected to be low. Thus, with major improvement and low attention allocation, as manifested by the marker, it may be advisable to challenge the client further. However, when the client does not improve sufficiently, it might be advisable to provide the client greater assistance.

To avoid bias, it seems advisable to evaluate the client's improvement during the session by goals, which are set a-priori in an objective and quantifiable manner. For example, the Goal Attainment Scale (GAS) is a general and accepted method for the evaluation of client improvement during the session, which is applicable for almost any type of rehabilitation and learning session. When the client's improvement is lacking and a cognitive barrier is encountered in a consistent manner, the exercise demands might be too challenging. In this case, it is advisable to moderate the challenge according to the client's abilities, for example, by reducing distractors, by providing an organizing strategy for the task—either general or task-specific or by changing the task altogether. If the barrier is constitutional and not exercise-dependent, medical treatment is also an option. If, on the other hand, an affective barrier (either avoidant or anxious) is encountered consistently without sufficient improvement during the session, additional interventions are possible. Significant avoidant or anxious responses to exercises hinder executive function and cognitive ability. Therefore, the various intervention strategies suggested above for cognitive barriers are applicable also for affective barriers. If the client learns an effective cognitive strategy to cope with the exercise, the stressing effect of the exercise may subside and the barrier may diminish.

Alternatively, or in combination, for some clients it might be preferable to focus on strategies directed at the affective barrier itself. As was stated above the affective barriers are often short-lasting and rapidly transient. The threat leads to an avoidant response, with attention and marker decrease to below the lower threshold, or to an anxious response, with attention and marker increase to above the higher threshold. But, within a minute or two, the client might feel that the exercise is manageable and not that threatening, with improvement in attention manifested by the return of the index toward the middle range. Thus, for some clients it might be enough to learn that new exercises might induce a transient affective barrier, which will subside shortly and performance will improve. In-fact, this advanced knowledge by itself might reduce the induced stress and the affective barriers.

Anxiety or avoidance might be more constitutional and the client might not be able to overcome them by adhering to the strategies suggested above. In such a case, it might be necessary to recommend to the client to take a break in the session for relaxation and possibly to teach the client relaxation techniques. In severe cases, where it seems that anxiety or avoidance are a major problem, medical interventions may improve the client's affective problem.

The Brain Engagement Index (BEI) is computed on the basis of template matching at the delta bandpass (1.5-4 Hz). The computation is based on measuring the number of occurrences of a pattern, which is composed of a sequence of large waves, lasting a few hundred milliseconds, followed by a sequence of small waves, also lasting a few hundred milliseconds. However, the inventor of the present invention recently discovered that it is not the precise pattern of waves that matters. Rather, it is the variability between epocs of greater delta power and epocs of lesser delta power. According to embodiments of the present invention an alternative index is presented—the Cognitive Effort Index (CEI). The CEI may be computed by dividing a several-seconds long EEG signal (for example—10-second segment) to a plurality of (for example—20 epocs of 500 milliseconds), computing the power of delta activity in each epoc, and the mean and standard deviation across epocs within a segment. Then the index is derived from the standard deviation and is normalized to the [0,1] range by dividing by a predefined factor (based on knowledge gained from multiple studies) or alternatively, for example by computing the standard deviation to mean ratio. Altogether the index could be normalized to the [0,1] range. We learned that, for example, if the standard deviation: mean ratio is greater than 1, it is likely to be due to a noisy sample, in which case no value is returned for this 10 second segment. FIG. 2E, to which reference is now made, illustrates how high variability among the 500 milliseconds epocs of the 10-second segments yields a higher CEI value in comparison with a lower variability segment, according to embodiments of the present invention.

The Goal Attainment Scale (GAS) principles may be used to evaluate the impact of the session upon a client performance. The GAS encourages the pre-session specification of objective and measurable functional goals—e.g., the distance the client would be able to walk at the end of the session, the number of correct objects the client would be able to name, etc. It is applicable and accepted throughout the rehabilitation and education professions. A frequently used version of the GAS is comprised of 5 points/levels (−2, −1, 0, +1, +2). Zero means the client reached the predefined expected performance by the end of the session, +1/+2 means the client outdid the therapist/teacher/trainer's expectation in terms of end of session performance and −1/−2 means the client improved less than expected or even deteriorated in performance by the end of the session. Embodiments of the present invention suggest differentiating between clients whose performance improves effectively over the session and clients whose performance does not improve, or perhaps even deteriorates. Therefore, instead of using a standard 5-point GAS range, embodiments of the present invention assign to a session performance improvement a scale of three-points: major improvement (beyond pre-session clinician's expectations), moderate improvement (accords with pre-session clinician's expectations) and no-improvement and potentially deterioration.

Further, the identified barriers and the client's performance dynamics may be combined over the session in order to derive meta-recommendations for the therapist/teacher/trainer. According to embodiments of the present invention three types of possible barriers (affective, cognitive or no-barrier) may be defined and three levels of performance dynamics rank may be defined (high which may be equivalent to GAS+1/+2, moderate which may be equivalent to GAS 0 and low which may be equivalent to GAS −1/−2). The recommendations that may be derived from the combination of performance dynamics levels and barrier levels (table 1), may provide a list of nine (9) interactions between performance dynamics and client's engagement barrier and respective recommendations related to adaption or changes to the client's practice. The recommendations may be directed to a therapist/teacher/trainer or may automatically change/adapt an exercise presented to the client.

TABLE 1

| | Performance | | |
|---|---|---|---|
| Barriers | Low performance | Moderate performance | High performance |
| Affective | (1) Low performance | (4) Moderate performance | (7) High performance |
| barrier | and affective barrier | and affective barrier | and affective barrier |
| Cognitive | (2) Low performance | (5) Moderate performance | (8) High performance |
| barrier | and cognitive barrier | and cognitive barrier | and cognitive barrier |
| No Barrier | (3) Low performance | (6) Moderate performance | (9) High performance |
| | and no barrier | and no barrier | and no barrier |

(1) When end-of-session performance is low (does not reach the expected level) and an affective barrier is noted, the following should be considered (a) the exercise level might be too demanding for the client and whether it should be moderated according to the principles suggested above for a cognitive barrier and (b) whether there should also be direct work upon the affective barrier, which seems to hinder performance further, according to the general lines suggested above for an affective barrier.

(2) When end-of-session performance is low and a cognitive barrier is noted, consider whether the exercise level might be too demanding for the client and should be significantly moderated, according to the principles suggested above for a cognitive barrier.

(3) When end-of-session performance is low and no barrier is noted, consider whether the client may have been well-engaged with some other thoughts, and the deficient performance may not represent true ability. It should be noted that usually when the client divides attention between the exercise and some unrelated thoughts, the attention and the attention index would drop due to the difficulty of maintaining effective divided attention. The fact that the index did not drop and therefore no barrier was noted means that the client may have in-fact successfully allocated the attention elsewhere and was not engaged with the exercise.

(4) When end-of-session performance is moderate (reaches just the expected level) and an affective barrier is noted, consider whether there should be work upon the affective barrier, which seems to hinder performance, according to the principles suggested above for an affective barrier.

(5) When end-of-session performance is moderate and a cognitive barrier is noted, consider whether the exercise level might be too demanding for the client and it should be moderated, according to the principles suggested above for a cognitive barrier.

(6) When end-of-session performance is moderate and no barrier is noted, the client seems to be struggling persistently with the challenge. However, consider a slight reduction of the exercise level, at-least temporarily, according to the principles suggested above for a cognitive barrier.

(7) When end-of-session performance is high (more than the expected level) and the affective pattern is noted, it does not seem to represent a barrier. Instead, it may represent allocation of attention to the exercise challenges and then relaxation when overcoming the challenges. In which case consider challenging the client even further.

(8) When end-of-session performance is high and the cognitive pattern is noted, it means that the exercise may be easy for the client. In which case consider challenging the client further.

(9) When end-of-session performance is high and no pattern of barrier is noted, it means that the client allocates significant attentive effort in order to advance performance successfully. In which case it is recommended to continue the current practice further and to gradually increase challenge.

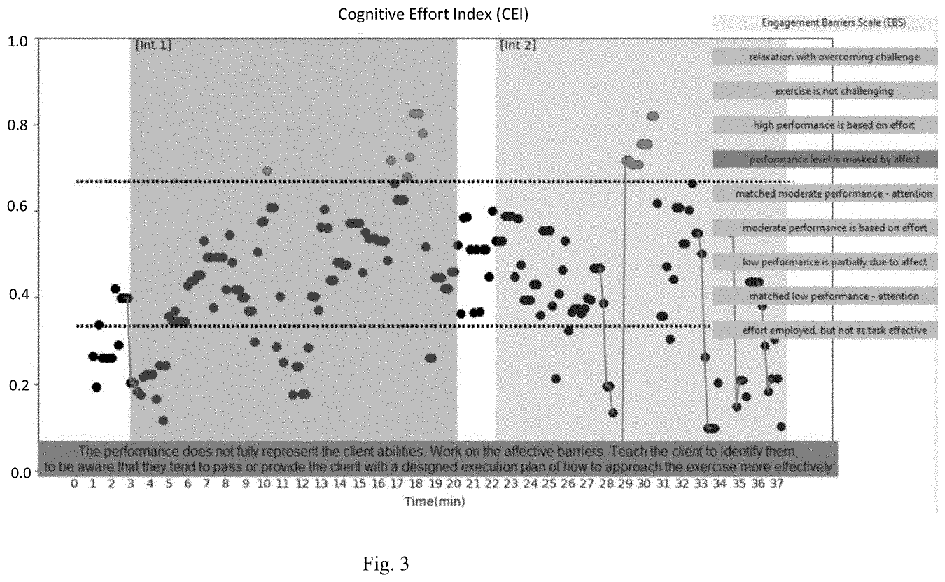

The tool described above may direct a therapist/teacher/trainer (or a computer-based therapeutic/teaching tool) to combine the performance dynamics level with the analyzed barrier to generate the automatic recommendation for the therapist/teacher/trainer. FIG. 3, to which reference is now made, presents a screenshot of a recommendation for moderate end-of-session performance and an affective barrier, according to embodiments of the present invention. The recommendation may be provided, in an alternative embodiment, in the form of computerized corrective instructions provided by a system (operative and built according to embodiments of the present invention) to a system that is adapted to present to the client with new task(s). The results of the attention index presented in FIG. 3 relate to two different interactions with the client, the left portion represents the results achieved in the first interaction and the right portion represents results achieved in the second interaction. On the right side of the screen there is a summarized list of optional system's conclusions based on the analyzed results, of which the one that is highlighted represents the conclusion matching the current case (“Performance level is masked by affect”). A detailed recommendation of a therapeutic step that should be taken is presented at the bottom of the screen (“The performance does not fully represent the client abilities. Work on the affective barriers. Teach client to identify them, to be aware that they tend to pass or provide the client with a designed execution plan of how to approach the exercise more effectively”). It would be apparent to those skilled in the art that such detailed recommendation may easily be converted to computerized set of instructions that when executed, for example by a computerized therapeutic system, may lead to re-configured task or challenge to the client, which will reflect the recommended change.

### Results—Case Reports

Below are presented three representative cases demonstrating the use of a tool operative according to embodiments of the present invention and the way it can combine the identification of barriers to attention engagement and the client performance. Further it is demonstrated how automatic recommendations generated by the tool at the end of a client's session may be used by the therapist/teacher/trainer in (or may provide automatic directions for) the following session to improve practice effectiveness. It is important to note that recommendations could also be derived during the session, to tune it in real-time, e.g. at the end on one exercise and prior to the start of the next one.

The three demonstrative case reports are of speech therapy for clients with aphasia and related dysfunctions following stroke. In the first case report the identification and practice of a cognitive barrier is discussed. It demonstrates how task level and therapist/therapeutic intervention need to be titrated when the client suffers from such a cognitive barrier in order to improve the client's rehabilitation. In the second case report the identification and treatment of an affective barrier is discussed. It demonstrates how the combination of task titration together with providing such clients with feedback that their true ability is hindered by their stressful condition can improve rehabilitation quite significantly. As clients understand their performance is hindered by stress and they can actually perform better, their stress tends to decrease and their performance immediately improves. This dynamic led to the typical rapid and significant improvement of the client presented in this second case report. The third case report presents an approach to a client with severe impairment who also suffers from both cognitive and affective barriers. It demonstrates that by addressing these two types of barriers effectively, it is possible to advance such clients' performance.

CASE 1: Background description of the participant. HM, 58 years old right-handed man, a native speaker of Hebrew. He was referred to our rehabilitation hospital following a left temporo-parieto-occipital hemorrhage that occurred two months previously. He had 12 years of education. Before the stroke, he was the owner of a locksmith shop and an artist, and had no premorbid language, reading, or writing disorders. At the time of the study, HM was 5 months post his stroke. He displayed mild Wernicke's aphasia with characteristic fluent spontaneous speech with occasionally semantic paraphasias, circumlocutions and word finding difficulties. His spontaneous speech clearly indicated that he could discuss only very simple and daily issues but failed to retrieve even very frequent imageable words. According to testing that was administered to him (see table 2 for his performance in various semantic and phonological tasks), he manifested a mild impairment in the semantic lexicon with a severe impairment in the phonological output lexicon.

TABLE 2

| | Spoken word to | Written word to | Repetition of | Picture |
|---|---|---|---|---|
| | picture matching | picture matching | non-words | naming |
| % correct | 70% | 80% | 94% | 0% |

Two sessions of speech therapy are described below. Both sessions were administered while the speech therapist was blinded to the attention marker during the treatments. At the end of each session the therapist reported HM's performance dynamics using the GAS scale. Based on her evaluation and the attention-based analyzed barrier, an automatic recommendation was generated for the therapist. Based on the recommendation following the first therapy session, the speech therapist re-evaluated her goals and the therapy procedures and tasks and planned accordingly the next session that took place on the following day.

First treatment: Goals and tasks. The main goal of the session was to evoke the retrieval of high frequency verbs, using two tasks. In the first task HM was required to retrieve a verb in a spoken sentence completion task. In the second task he was introduced to action pictures and was required to describe the pictures using simple sentences (subject-verb-object). Results and recommendations. HM seemed alert and responsive during the whole session. He was cooperative with the therapist and it seemed that he is making genuine efforts to retrieve the words. However, his performance throughout the session was low and specifically lower than the expected level. Based on her expectations, the therapist evaluated HM performance in both tasks as GAS=−1, namely, a performance below initial expectation.

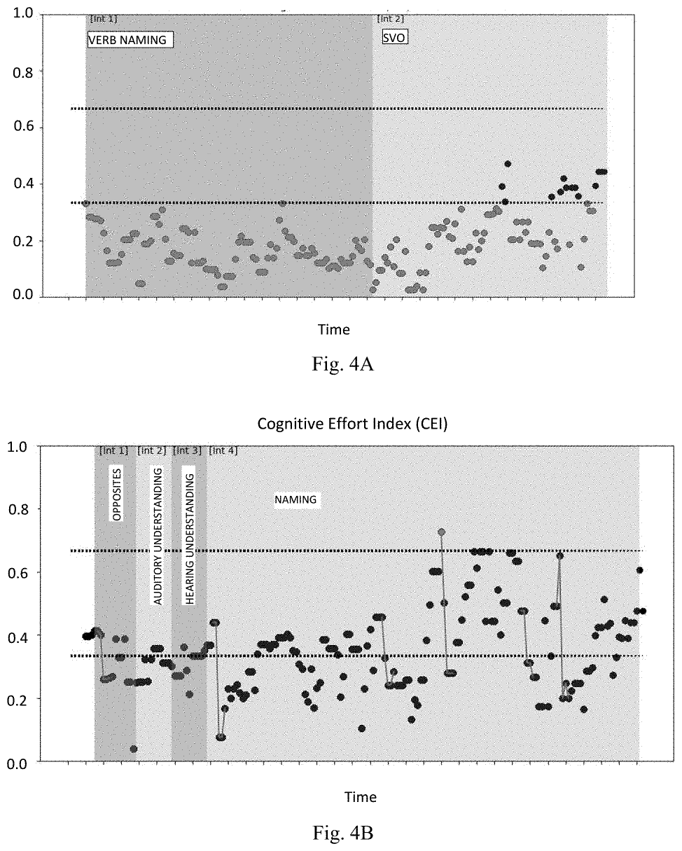

FIG. 4A, to which reference is now made, presents the attention index dynamics during a session. According to embodiments of the present invention. As can be seen, the vast majority of the points are consistently below the low threshold. In the first task—verb retrieval (dark grey, left portion) the client index was below the low threshold throughout the task. In the second task—action picture description (light grey) the client index was below the low threshold 82% of the task duration. This pattern, combined with HM's low functional performance, is consistent with a cognitive barrier to engagement. Despite the apparent responsiveness, the combination of this pattern and the low performance may mean that HM was unable to attend effectively to the exercise. This is often because the level of the tasks might be too demanding for him and should be moderated (see also in Table 1—low performance (1st column), cognitive barrier (2nd row), which leads to the automatic recommendation no. (2) presented above, to consider whether the exercise level might be too demanding for the client and should be significantly moderated, according to the principles suggested above). Given the dynamics of the first treatment, different goals were set, and lower-level tasks were administered.

Second treatment. Goals and tasks. The main goals of this session were the followings: a) to be able to retrieve at least 75% of very frequent nouns, adjectives and adverbs in a sentence completion task. b) to achieve at least 90% success in spoken and written word-to-picture matching task out of 8 semantic related distractors. c) to enhance the use of compensatory strategies to convey the meaning of words in a simple picture naming task that includes very frequent high imageable words and to follow external semantic or phonological cues given by the therapist. For the first goal HM was instructed to complete a given spoken sentence. The therapist introduced a spoken sentence that contained a noun, adjective or adverb (“One house is big but the other house is . . . ”) and HM was requested to complete the sentence with the opposite word. For the second goal, sets of eight semantic high frequency related pictures were introduced to HM and he was requested to point at the picture that matched the spoken or written word. For the third goal HM was confronted with colored pictures of very frequent nouns and was requested to name the pictures or to use compensating or enhancing strategies whenever he was confronted with a word finding difficulty, strategies that may be helpful to evoke the target words-related gestures, retrieval of a related word, description of the target word. Furthermore, he was instructed to follow external semantic or phonological cues given by the therapist whenever he failed to retrieve words independently.

Results and recommendations. Like the previous session, in the current session HM also appeared alert and cooperative. Yet, unlike the previous session, he performed much better. In the first task he performed according to the speech therapist's expectations and therefore she evaluated HM's performance as GAS=0. He successfully retrieved about 75% of the opposite words and in the words that he failed, he benefited from phonological cues that were offered by the therapist. In the second and third tasks, spoken word and written word matching tasks, he successfully and effortlessly chose the correct target and therefore he received a GAS score of +1. In the final task, picture naming, his performance was much better than expected, although for most of the pictures he failed to retrieve the words immediately, still self-use of strategies and additional cues from the therapist finally led to accurate naming of all the pictures—GAS=+1.

FIG. 4B, to which reference is now made, presents the attention index dynamics during the client's session, according to embodiments of the present invention. As can be seen, there are 4 intervals—first left (dark grey) reflects the index dynamics during the retrieval of opposites in a sentence completion task. In this task the performance was according to expectations (GAS=0). At the beginning of the task there was a rapid drop to below the lower threshold, which may reflect a temporary affective barrier of avoidance, which may have led to the moderate success (see also in Table 1—moderate performance (2nd column), affective barrier (1st row), which leads to the automatic recommendation no. (4) presented above, to consider whether there should be work on the affective barrier, which seems to hinder performance, according to the principles suggested in section 4 of the introduction for an affective barrier). However, soon enough HM overcame this avoidance and then stayed in the middle range for 31% of the task. The second and the third intervals (light and dark grey respectively) present the attention index dynamics during the spoken word-picture matching task and written word-matching task. In these tasks the performance was much better than expected (GAS=+1) and the points are mostly below the low threshold, 75% of the time in the spoken word-to-matching task and 83% of the time in the written-to-picture matching task. This pattern, combined with HM's high functional performance indicates an easy task, which should probably be replaced with a more demanding and challenging task (see also in Table 1—high performance (3rd column), reduced cognitive effort (2nd row), which leads to the automatic recommendation no. (8) presented above, to consider challenging the client further). Finally, the light grey long interval presents the attention index dynamics during the picture naming task (with self and external cueing) in which his performance was also higher than expected (GAS=+1). In this task there are four rapid drops to below the lower threshold. This repetitive pattern, indicating affective dynamics, which may indicate an exercise that is too easy or too much assistance given by the therapist, leading to rapid relaxation (see also in table 1—high performance (3rd column), positive affective response (1st row), which leads to the automatic recommendation no. (7) presented above, to consider challenging the client further). It appears that while the first session tasks were too difficult for HM and therapist guidance was not enough for HM, the second session may have been too easy. The challenge is to select and monitor a more appropriate level of challenge and assistance. For this aim, the monitor could also be used in real-time during a session.

CASE 2—overcoming the affective barrier. Background description of the participant. EB, 79 years old right-handed man, a native speaker of Hebrew. This was not his first stroke. About 3 months prior to the recent stroke, he had a right parietal hemisphere infarct and according to MRI he had additional old bilateral cerebellum and corona radiata infarcts. Prior to the current stroke he was an active physician in a private clinic. At the time of the study, EB displayed a very severe conduction aphasia with characteristic fluent spontaneous speech, occasionally phonological paraphasias and phonological approximations and severe word finding difficulties. He seemed very frustrated and manifested difficulties conveying even very simple and daily ideas including personal basic information. According to testing administered to him (see also: Table 3 for his performance in various tests), he manifested preserved auditory and written word level and sentence level comprehension, indicating preserved semantics and preserved comprehension of syntax. Repetition was moderately to severely impaired. It seemed that the main source of his deficit lied in the activation of phonological and orthographical representations from the phonological and orthographical output lexicons with an additional deficit in the phonological output buffer.

TABLE 3

| | | | | Comprehension | | |
|---|---|---|---|---|---|---|
| | Spoken | Written | | of simple and | | |
| | word to | word to | Repetition of | syntactically | | Writing |
| | picture | picture | words and non- | complex spoken | Picture | to |
| | matching | matching | words | sentences | naming | dictation |
| % | 100% | 100% | Words - | 100% | 10% | 0% |
| Correct | | | 40% | | | |
| | | | Non- | | | |
| | | | words - | | | |
| | | | 37% | | | |

Three sessions of speech therapy are discussed in the following paragraphs. These were sessions that took place two weeks after the client arrived at the rehabilitation centre, namely after one session of evaluation and only a few sessions of treatment. The sessions reported below were administered to him while the therapist was blinded during the treatments to the computations of the attention index marker. At the end of each session the therapist reported EB's performance dynamics using the GAS scale. Based on her evaluation and the attention index computation of the analysed barrier, an automatic recommendation was generated for the therapist. Based on the recommendations following each therapy session, the speech therapist re-evaluated her goals and the therapy procedures and tasks and planned the next session that took place a few days later accordingly.

First treatment: Goals and tasks. Given the very severe deficit of retrieval of the phonological representation of words from the phonological output lexicon, the main goal of the session was to evoke the retrieval of words in a highly supportive environment. The session started with a short conversation that EB initiated about events that occurred the day before while the therapist encouraged him to use his supportive communication aid (small notebook with written words and sentences) or gestures or paraphrases or even writing or drawing whenever he encountered a word finding difficulty. In the next task EB was asked to name pictures of very high frequency words and here again he was encouraged to use gestures or to produce or write relevant word definitions whenever he failed to retrieve the word. Next, he was requested to retrieve words that are semantically related to a specific topic (“think of words that are related to your profession”) and finally, after a brief spontaneous conversation, he was asked to complete spoken sentences with common nouns, verbs, adjectives or adverbs.

Results and recommendations. Apparently, the tasks were very difficult for EB. His performance through all the session was low, and specifically lower than the expected level for most of the tasks. Based on her expectations, the therapist evaluated EB's performance in the short conversation as expected, namely GAS=0; and in all other tasks as GAS=−1, namely, less than expected performance.

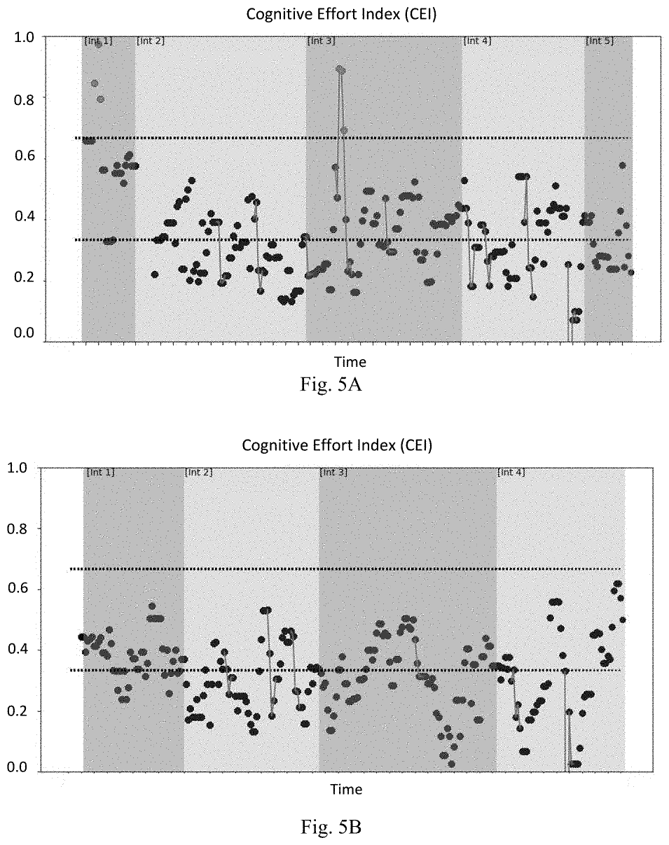

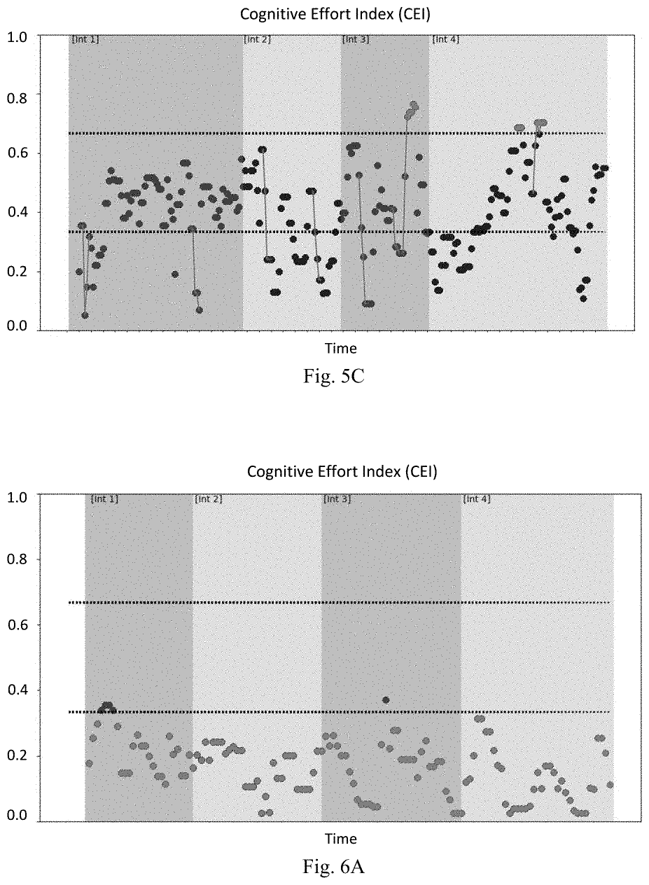

FIG. 5A, to which reference is now made, presents the attention index dynamics of the session, according to embodiments of the present invention. Overall, during five different exercises there were nine episodes of rapid drop to below the lower threshold and one rise to above the higher threshold, which may point to an affective barrier. These patterns were presented in the various tasks and thus seem to indicate a general response of EB to speech therapy, beyond task specifics. Taken together with the low performance this seems to indicate a severe affective barrier, namely EB's performance seems severely hindered by stress, which is possibly induced by the challenges of speech therapy (See also in Table 1—low performance (1st column), affective barrier (1st row), which leads to the automatic recommendation no. (1) presented above, to consider whether the exercise level might be too demanding for the client and whether it should be moderated, according to the principles suggested in section 4 of the introduction for an affective barrier).

Second treatment. Given the dynamics of the first treatment and particularly the affective barrier that was evinced, a different approach was set to reduce the avoidant and anxious responses as much as possible. In the second session it was decided to continue with a highly supportive environment that was manifested in two main ways: first, to offer EB various types of immediate cues, semantic and phonological cues, when confronted with a word finding difficulty and not to wait for exhaustive trial and error self-attempts that usually led to frustration and possibly to avoidance or anxious response; second, to show him the attention index dynamics of the former session, and specifically to instruct him to pause, to breathe deeply and wait whenever he felt that he was about to fail to retrieve the word and just to let it go.

Goals and tasks. The main goals of this session did not dramatically change. There was hope to achieve better performance in all the tasks and less avoidant and anxious responses in the attention index dynamics. The first task included a conversation on the day before that he initiated. The second task was a sentence completion task that included the retrieval of very high frequency nouns, adjectives, adverbs and verbs. In the third task he was requested to describe pictures in simple sentences with action words and he was instructed to accompany his descriptions as much as possible with relevant gestures. In the fourth task he was given a syllable, such as “bi” and was requested to produce any word that came to his mind which began with that syllable.

Results and recommendations. Unlike the previous session, EB made fewer trial-and-error attempts since the therapist offered immediate cues and instructed him to pause and breathe whenever the self-efforts did not lead to correct performance. The therapist evaluated his performance in spontaneous speech, as expected, namely GAS=0; In the following task (sentence completion) as GAS=1; in the sentence description task as GAS=0; in the final task, GAS=0.

FIG. 5B, to which reference is now made, presents the attention index dynamics of the session, according to embodiments of the present invention. Like the first session, here as well there were episodes of rapid drops to below the lower threshold that reflected a temporary affective barrier of avoidance. Still there were only 6 rapid changes (compared to 10 in the previous session) without any rises above the upper threshold. However, despite the improvement, the affective barrier was still not overcome and the attention index in about half of the session was still below the lower threshold (51%).

Third treatment: In the third session it was examined whether the tendency of improvement in the affective barrier that had been achieved in the second treatment would continue. For this aim, the setting of the third treatment was very similar to that of the second one except for the fourth task, as EB wanted to end the session with a short conversation on plans that he had for later in the day. Like the second treatment the attempt was to minimize the affective barriers by pausing, breathing and by using various compensating strategies to overcome failures in conveying his messages and in the naming tasks.

Results and recommendations. Like the previous session, the therapist evaluated his performance in spontaneous speech, as expected, namely GAS=0; yet on the other tasks, sentence completion and simple sentences production he received a score of GAS=+1.

FIG. 5C, to which reference is now made, presents the attention index dynamics during the session, according to embodiments of the present invention. Here again there were five rapid drops to below the lower threshold and also two rises to above the higher threshold. Still these deviations were short lasting (generally less than a minute) and the tendency to overcome the affective barrier more rapidly was even stronger, with longer time in the middle range level engagement (63%) compared to the second (49%) and the first (45%) sessions. These dynamics, combined with performance on the majority of exercises, which was above therapist expectations (GAS>0), implies that this approach of trying to overcome the affective barrier might be productive for the client and, in-fact, the sharp drops with a limited low period thereafter may indicate the client's relaxation after success (see also in Table 1—high performance (3rd column), positive affective response (1st row), which leads to the automatic recommendation no. (7) presented above, to consider challenging the client further). In which case, it might be possible to increase the demands in the next session.

CASE 3: Background description of the participant. SG, 64 years old right-handed man, a native speaker of Hebrew. He was referred to rehabilitation following an extensive infarct in the left middle cerebral artery that occurred about 5 weeks earlier. Before the stroke he worked as a driver. At the time of the study, SG displayed Global aphasia and severe phonatory, bucco-facial and speech apraxia. He was totally mute, though it was possible to hear his voice in spontaneous coughing and yawning. He was unable to repeat even single vowels and to name objects and pictures. Also, his auditory and reading comprehension were severely impaired. He manifested moderate difficulties in auditory and written word picture matching tasks of three words—the target and two semantically and phonologically unrelated distractors and failed to do so in larger sets or in semantically related sets. In addition, he had some difficulty in associating pictures to one of two semantically unrelated categories (vehicles versus fruits) and to associate between gestures and objects. We therefore surmised that the loci of the impairment lie at the conceptual semantic level and the semantic lexicon in addition to his severe phonatory, bucco-facial and speech apraxia.

The main short-term goals were to achieve voluntary voice production and to improve his conceptual and lexical semantic capabilities. Therefore, the treatment sessions focused on sorting pictures into semantic categories; auditory and written word matching with very limited and semantically distant distractors and effortful trials to produce voice. At the time of the study, SG received 5 thirty minutes treatment sessions per week with only very mild improvement.

Three treatment sessions were monitored. These sessions took place one and two weeks apart. In between he continued to receive regular treatments. The first monitored treatment session took place about 8 weeks post stroke. Like the former case reports the therapist was blinded during the treatments to the attention marker and after each session she reported the participant's performance dynamics using the GAS scale.

First treatment: Goals and tasks. Along the regular treatment sessions, the therapist was under the impression that SG experiences very challenging and frustrating moments in his unsuccessful voluntary vocalizations attempts. Therefore, she decided to focus on vocalizations but only after he had experienced some success on other tasks. The rationale was that if he experienced success on other tasks, it would hopefully encourage and engage SG in the task that seemed to be the most frustrating task for him—voluntary vocalization. To this aim, 3 tasks were administered prior to the vocalization task. The first task was a semantic conceptual task—odd picture out task—finding the odd picture out of 4 pictures—three of them from the same semantic category. Given the relatively mild-moderate conceptual impairment, the prediction was that SG would perform relatively well on this task. The second and the third tasks were lexical semantic tasks, which involved auditory and written word-to-picture matching tasks using the target word and two semantically and phonologically unrelated distractors. The prediction was that his performance would be relatively good (about 80% success). The final and presumably the most challenging target was to achieve some voluntary vocalizations trying to imitate the therapist vocalizations of vowels, while the therapist utilized manipulation of moderate external pressure on SG's diaphragm.

Results and recommendations. As expected, SG performed well in the conceptual, odd picture out task and therefore his performance was rated as GAS=0. His performance on the auditory and the written word to matching tasks was surprisingly good—with almost no errors; therefore he was rated: +1. Yet he succeeded to vocalize only when the manipulation was utilized, as expected, and therefore his performance on the vocalization task was rated as GAS=0.

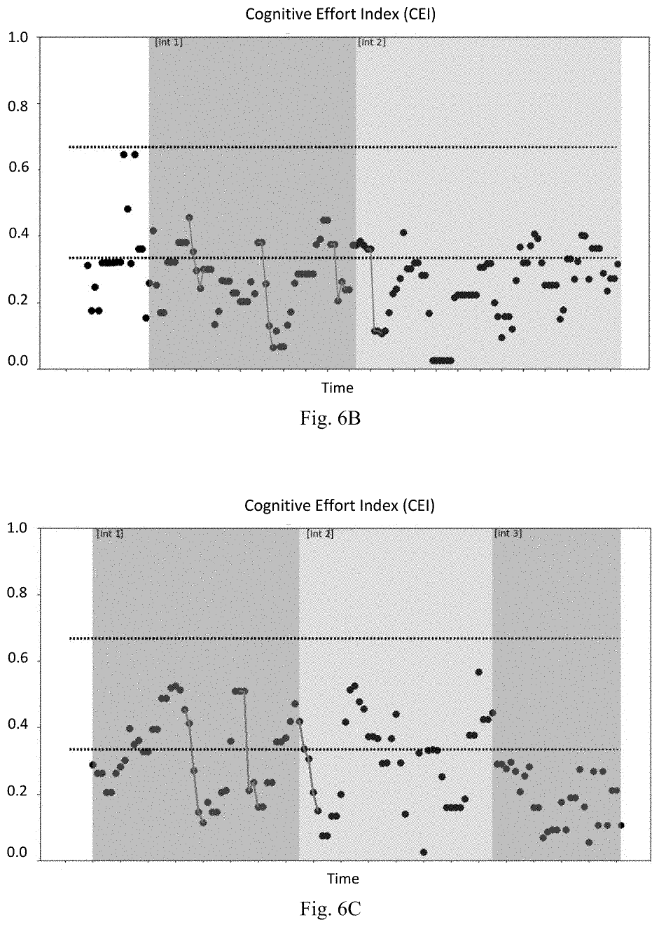

FIG. 6A, to which reference is now made, presents the attention index dynamics during the session, according to embodiments of the present invention. The leftmost interval presents the attention index dynamics at the beginning of the session, during which there were attempts of interactions between the therapist and SG. The second interval presents the dynamics of the conceptual task. The third interval presents the dynamics of the lexical semantic auditory and the written word to picture matching tasks and the fourth interval presents the vocalization task. As can be seen, the points are consistently below the low threshold during the entire session: 100% of the time in the conceptual and the voicing tasks and 99% of the time in the lexical semantic tasks. This pattern of low engagement (and therefore low cognitive effort) during the entire session combined with SG relatively good performance was unexpected and surprising. It seems that the tasks did not challenge him enough, which was difficult to predict in the first place, due to his significant impairment (see Table 1—high performance (3rd column), reduced cognitive effort (2nd row), which leads to the automatic recommendation no. (8) presented above, to consider challenging the client further). Therefore, the therapist re-evaluated the goals and tasks for the following treatment session so that they would be more challenging and engaging.

Second treatment: Goals and tasks. Given SG's success in the auditory and written word to picture matching (with two distractors) in the first monitored session and in consecutive sessions and the attention index dynamics during the first session, the goal of the second monitored session was to achieve some success on more demanding tasks. Therefore, auditory simple sentences were introduced to SG and he was requested to choose the matched picture out of three pictures—the target and two foils. In all pictures the agent argument was constant but each time the verb was replaced (“show me the picture of the man eating”; “show me the picture of the man drinking”; “show me the picture of the man shaving”). Given that the difference between the sentences was only in the verb and that there were only two distractors, we expected that the performance would be above chance. The second goal was that SG would hopefully be able to produce voluntary voice following an imitation of laughter. In addition, he was requested to blow out a candle by saying “f”, imitating the therapist, hoping to achieve the production of that phoneme.

Results and recommendations. Unlike what the therapist expected, SG failed on the auditory sentence-to-picture matching task and therefore his performance was rated as GAS=−1. On the other hand, he performed much better than expected on the vocalization tasks: he succeeded in producing some voluntary voices following a laughter that was triggered by the therapist and produced the “f” sound while blowing out the candle. More than that, he succeeded in producing some more “f”s after the candle was out of his sight. His performance on these tasks was rated as GAS=+1.

FIG. 6B, to which reference is now made, presents the attention index dynamics during the session, according to embodiments of the present invention. As can be seen, there were two intervals—first left (in dark grey) reflects the attention index dynamics during the auditory sentence-to-picture matching task. In this task though SG was more engaged than the first treatment (26% of the time he was in the middle range and 74% in the lower range compared to 100% in the lower range in the first treatment session). Still, the performance was below expectation (GAS=−1). The low performance can be ascribed to an affective barrier, as is manifested by the 3 rapid drops to below the lower threshold, which seem to represent an avoidance response (See Table 1—low performance (1st column), affective barrier (1st row), which leads to the automatic recommendation no. (1) presented above, to consider whether the exercise level might be too demanding for the client and whether it should be moderated, according to the principles suggested in section 4 of the introduction for an affective barrier). In the second vocalization task SG was also more engaged compared to the first treatment (21% of the time he was in the middle range). But generally, he was still mostly in the lower range. This pattern of reduced attentional effort, taken together with the surprisingly good performance compared to the therapist's expectation (GAS=+1) in this main treatment goal of vocalization may mean that the client was able to perform significantly better than originally expected (see Table 1—high performance (3rd column), reduced cognitive effort (2nd row), which leads to the automatic recommendation no. (8) presented above, to consider challenging the client further), and could be challenged more. However, the pattern of avoidance that was manifested along the auditory sentence to picture matching task implies that his performance was highly affected by the affective barrier, possibly because the task was too demanding and, as a result, stressful for him. Therefore, to overcome the affective barrier, the therapist decided to implement a different approach to reduce the avoidant response as much as possible and to continue to encourage vocalization in more automatic settings.

Third treatment: Goals and tasks. A new goal was set for this monitored session. The goal was that SG would be able to convey/transact some information in an interaction with a communication partner (the therapist). In this task the therapist posed some questions to SG such as: “Where do you live? How long have you been married? SG was requested to use picture communication aid boards to answer the questions. The second goal was to achieve a relatively good performance on a semantic-lexical task using auditory and written word-to-picture matching tasks. Unlike the former trials, this time all the pictures were of Jewish religious articles. Given that SG is an observant Jew, it was assumed that these articles (prayer shawl, skull cap, the prayer book and more) would be emotionally engaging and might lead to a more accurate performance even with a larger set of foils. Following that task, the goal of the third task was to achieve vocalization while trying to sing the traditional Chanuka lighting candle prayer. Given that the session took place during the Chanuka holiday, the therapist assumed that the excitement and the importance of the religious ceremony for SG might engage him to be able to produce some voluntary melodic sounds.

Results and recommendations. The performance on all the tasks was much higher than expected. On all tasks he was rated GAS=+1. He used the picture boards quite efficiently and conveyed relevant responses to the questions that were posed to him by pointing to the correct pictures, responses that manifested understanding of both the questions and the purpose of the communication boards. He performed flawlessly on the auditory and written word to picture matching task even when there were the target picture and 7 distractors. Finally, he succeeded in vocalizing the tune and even to pronounce partial fragments of the prayer together with the therapist. This was the first time since the stroke that he succeeded in vocalizing and producing some meaningful sounds.

FIG. 6C, to which reference is now made, presents the attention index dynamics of the session according to embodiments of the present invention. As can be seen, there are three intervals—the first left reflects the attention index dynamics during the interaction with the therapist that was aided by the communication boards. In this task it was the first time that SG was fully engaged for half of the time (47% of the time he was in the middle range). There were 2-3 episodes of rapid drops to below the lower threshold (one of the episodes took place at the end of the interval), which taken together with his good performance (GAS=+1), seem to represent relaxation after successful performance (see Table 1—high performance (3rd column), positive affective response (1st row), which leads to the automatic recommendation no. (7) presented above, to consider challenging the client further). The second interval reflects the dynamics during the auditory and written sentence to picture matching tasks, pictures of religious articles. The episode of rapid drop at the beginning of the interval might relate to relaxation after the communication boards task, or alternatively may relate to an initial and rapidly transient avoidance response to the new task. But overall SG manages to stay in middle range of engagement for 41% of the task duration. This was also manifested in his performance (GAS=+1), compared to similar tasks in previous sessions. In the last task—singing the traditional Chanuka candle lighting prayer, he did very well (GAS=+1) and according to the attention index dynamics he was not engaged at all—100% of the time he was in the lower range. It seems then, he at-least has the ability to vocalize during an automatic procedure, since this is a very familiar prayer to him and therefore very easy for him and not challenging. Though this task yielded relatively good performance, especially compared to the previous sessions, based on the attention index dynamics (see Table 1—high performance (3rd column), reduced cognitive effort (2nd row), which leads to the automatic recommendation no. (8) presented above, to consider challenging the client further) it seems that SG might be challenged further in terms of vocalization. This is quite informative considering his near mutism at baseline. It might be possible to use the Chanuka prayer as anchor and try to increase the complexity of demand gradually, with vocalizations which are less habitual for SG.

Summary of the case reports: The three case reports presented above demonstrate how the tool operative according to embodiments of the present invention is utilized to combine the attentional engagement monitoring for cognitive and affective barriers and the clients' performance in order to derive automatic recommendations for the therapist/teacher/trainer and how these recommendations are implemented to obtain significantly better rehabilitation of the core impairments. While the demonstrations were for speech therapy, the application is general throughout rehabilitation and education and we have already treated numerous clients undergoing physical and cognitive therapies with the tool. In these demonstrations the use of the tool was at the end of the session and toward the next session, which is easier, as the therapist/teacher/trainer is not required to attend to the monitor during the session. Nevertheless, therapists/teachers/trainers who acquire skill with the tool can use the tool in real-time during the session to switch or tune the on-going tasks, especially if after previously monitored sessions, the barriers impeding the specific client's performance had been established and the impact of interventions upon them could be evaluated in real-time. In alternative embodiment, as discussed above, the recommendations for changes in the client's exercise of the next treatment may be automatically provided by a system operative according to embodiments of the present invention directly to a computerized treatment unit adapted to present a client with treatment exercises configurable in accordance to treatment recommendations provided by a system of the present invention.

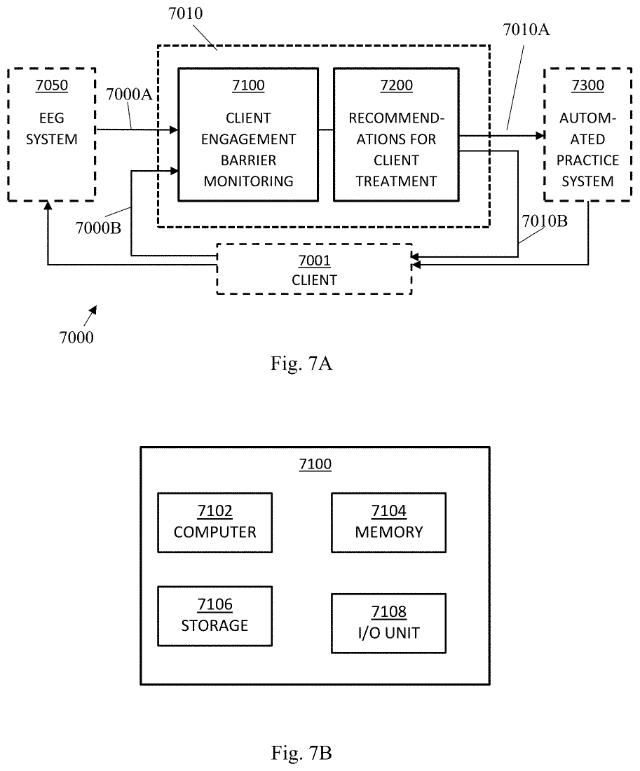

Reference is made now to FIGS. 7A and 7B present schematic block diagram of system 7000 for providing of practice recommendations based on barriers to client engagement and of computing unit 7010 adapted to compute the barriers and to provide practice recommendations, respectively, according to embodiments of the present invention. System 7000 schematically describes a practice environment for client 7001 that may comprise an EEG/EMG system 7050, a computing unit 7010 and an automated exercise/task unit 7300. Computing unit 7100 may be adapted to receive EEG/EMG electrophysiological signals 7000A and indications 7000B of the success of client 7001 in performing practice tasks. Computing unit 7010 may be adapted to perform monitoring of the engagement barriers of the client in performing tasks, and to provide practice recommendations (7010A, 7010B) for next practice session(s) based on the computed engagement barriers and the level of success of the client in performing the last task. EEG/EMG system 7050 may be any known system adapted to provide electrophysiological signal. Signal 7000B indicative of the success of client 7001 in performing practice tasks may be any feedback signal, such as evaluation of the success provided by the therapist/teacher/trainer or a signal received from a computerized practice system. Practice recommendations 7010A may be adapted to be received by a computerized practice recommendations unit 7300 that may initiate practice task for client 7001. Practice recommendations 7010B may be provided to the therapist/teacher/trainer working with client 7001.

Computing unit 7100 may comprise computer 7102, memory unit 7104, storage unit 7106 and I/O unit 7108. Computer 7102 may be, for example, a central processing unit processor (CPU), a chip or any suitable computing or computational device. Memory unit 7104 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory unit 7104 may be or may include a plurality of, possibly different memory units. Storage unit 7106 may be or may include, for example, a hard disk drive, a floppy disk drive, a Compact Disk (CD) drive, a CD-Recordable (CD-R) drive, solid state drive (SSD), solid state (SD) card, a Blu-ray disk (BD), a universal serial bus (USB) device or other suitable removable and/or fixed storage unit. Content may be stored in storage unit 7106 and may be loaded from storage unit 7106 into memory unit 7102 where it may be processed by computer 7102. Storage unit 7106 may be a non-transitory storage unit. I/O unit 7108 may be adapted to receive electrophysiological signal from a EEG/EMG system and signals indicative of the success of client 7001 in performing his tasks.

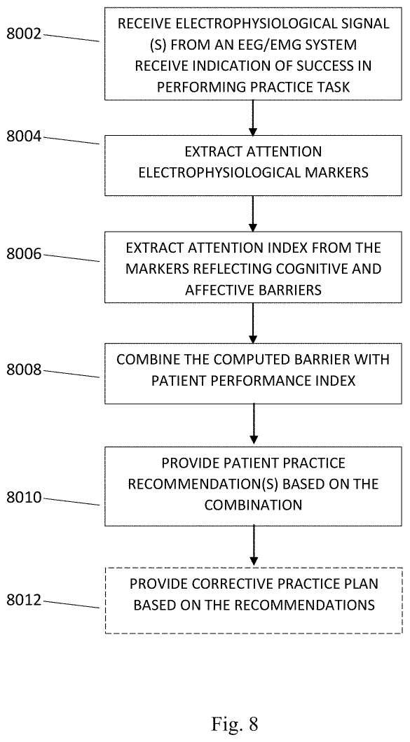

Reference is made now to FIG. 8, which is a top-level schematic flow diagram depicting a method for real-time monitoring barriers to client's engagement and for providing practice recommendations, according to embodiments of the present invention. Electrophysiological signal may be received from a client, e.g. from an EEG or EMG system and indication of the success of the client in performing a current practice task (step 8002). Electrophysiological markers indicative of the level of attention of the client may be extracted from the electrophysiological signal (step 8004). Attention index of the client in performing practice task may be computed and attention barrier may be defined based on the computed attention index: affective barrier, cognitive barrier or no barrier (step 8006). The computed attention barrier may be combined with the performance index to yield a table of optional practice recommendations (step 8008). Practice recommendation may be provided based on the combination of the attention barrier and the performance index (step 8010). Practice recommendations may be provided to the client by therapist/teacher/trainer, or, in case the practice is carried by a computerized practice system (such as unit 7300 of FIG. 7A), the practice recommendations may be provided by a computerized practice system that may be adapted translate the practice recommendations into a practice task.

It would be apparent to those skilled in the art that embodiments of the present invention may be useful, alternatively or additionally, also in additional fields. One example is during evaluation session. For example: suggesting relaxation after overcoming challenge, when an exercise is not challenging, understanding when high performance is based on effort, understanding when performance level is masked by affect, understanding that moderate performance ability correctly reflects the situation based on effort level and attention allocation, understanding when effort was allocated, but not effectively to the task. A different filed in which embodiments of the invention may be useful is in a session in which the client is exposed to media: relaxation after overcoming challenge—the media is interesting and enjoyable (first there is an increase of attention with the media induced challenges and then relaxation with overcoming the induced challenges); high performance is based on effort—the media is challenging, but within reach, for the client's understanding; performance level is masked by affect—the media induces discomfort, which moderately hinders its understanding; true moderate performance ability—the media is somewhat too challenging for the client; moderate performance is based on effort—the media is complex for the client, despite his/her effort; low performance is partially due to affect—the media is complex for the client and also induces discomfort; true low performance ability—the media is very complex for the client's understanding; and effort was allocated, but not effectively to task—check whether the client was engaged with the media.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for providing practice recommendations during or following a practice session, comprising:
receiving at least one electrophysiological signal of a client from an EEG system or an eye tracking system during the practice session;
receiving indication of the success of the client in performing a task during the treatment session;
extracting electrophysiological markers for attention/engagement/effort of the client during the performance of the task;
extracting client engagement barrier types from the electrophysiological markers;
classifying client engagement barrier types to one of: affective barrier, cognitive barrier and no barrier;
classifying the success level of the client in performing the task to one of a plurality of discrete success levels; and
providing practice recommendation for a future practice based on the specific success level and on the identified attention barrier.

2. The method of claim 1, wherein the plurality of discrete success levels comprise: low performance, moderate performance and high performance.

3. The method of claim 1, wherein the extracting of client engagement barriers from the electrophysiological markers comprises extraction of an attention/engagement/effort index.

4. The method of claim 3 wherein the extraction of an attention/engagement/effort index comprises dividing the electrophysiological signal into a plurality of segments and dividing each of the segments into a plurality of epocs.

5. The method of claim 4 wherein the duration of each of the plurality of the segments is in the range of seconds to tens of seconds and the duration of each of the epocs is in the range of hundreds of milliseconds to seconds.

6. The method of claim 5 wherein the duration of each of the plurality of the segments is 10 seconds and the duration of each of the epocs is 500 milliseconds.

7. The method of claim 6 further comprising excluding epocs in which the signal deviation is above a predefined level, to remove noisy epocs.

8. The method of claim 7 further comprising assigning power index to each of the remaining epocs according to the average absolute amplitude of the signal in each epoc of the remaining epocs and normalizing the power index to a normalized range.

9. The method of claim 8 further comprising identifying attention barrier type associated with the received signal based on normalized power index dynamics and the relation between the normalized power indices to a lower threshold and to a higher range in the normalized range.

10. A system for providing practice recommendations during or following a practice session, comprising:
a computing unit adapted to receive at least one electrophysiological signal of a client from an EEG system or an eye tracking system during the practice session and indication of the success of the client in performing a task during the practice session, the computing unit comprising:
a central processing unit (CPU);
a memory unit;
a non-transitory storage unit; and
an input/output unit,
wherein the CPU is adapted to perform executable code loadable from the memory unit and/or the storage unit,
wherein the input unit is adapted to receive the at least one electrophysiological signal of a client from an EEG system during the practice session and the indication of the success of the client in performing the task during the practice session, and
the output unit is adapted to provide practice recommendations based on the received one electrophysiological signal of a client from an EEG system during the practice session and received indication of the success of the client.

11. The system of claim 10 further adapted:
to extract electrophysiological markers for attention of the client during the performance of the task;
to extract client engagement barrier types from the electrophysiological markers;
to classify client engagement barrier types to one of: affective barrier, cognitive barrier and no barrier;
to classify the success level of the client in performing the task to one of a plurality of discrete success levels; and
to provide practice recommendations for a future practice based on the specific success level and on the identified attention barrier.

12. The system of claim 11 wherein the plurality of discrete success levels comprise: low performance, moderate performance and high performance.

13. The system of claim 11 wherein the extracting of client engagement barriers from the electrophysiological markers comprises extraction of an attention/engagement/effort index.

14. The system of claim 13 wherein the extraction of an attention/engagement/effort index comprises dividing the electrophysiological signal into a plurality of segments and dividing each of the segments into a plurality of epocs.

15. The system of claim 14 wherein the duration of each of the plurality of the segments is in the range of seconds to tens of seconds and the duration of each of the epocs is in the range of hundreds of milliseconds to seconds.

16. The system of claim 15 wherein the duration of each of the plurality of the segments is 10 seconds and the duration of each of the epocs is 500 milliseconds.

17. The system of claim 16 further comprising excluding epocs in which the signal deviation is above a predefined level, to remove noisy epocs.

18. The system of claim 17 further comprising assigning power index to each of the remaining epocs according to the average absolute amplitude of the signal in each epoc of the remaining epocs and normalizing the power index to a normalized range.

19. The system of claim 18 further comprising identifying attention barrier type associated with the received signal based on normalized power index dynamics and the relation between the normalized power indices to a lower threshold and to a higher range in the normalized range.
